(19) United States
(12) Patent Application Publication
(10) Pub. No.: US 2003/0171817 A1
(43) Pub. Date: Sep. 11, 2003

(54) Cotyloid insert for total hip prosthesis

(76) Inventors: Andre Rambert, Lyon (FR); Jean-Paul Carret, Lyon (FR); Jean-Paul Cisterne, Saint Eloi (FR); Michel-Henri Fessy, Millery (FR); Emmanuel Baulot, Dijon (FR); Frederic Farizon, Saint Chamond (FR)

Correspondence Address: CANTOR COLBURN, LLP, 55 GRIFFIN ROAD SOUTH, BLOOMFIELD, CT, 06002

(21) Appl. No.: 10/240,959

(22) PCT Filed: Apr. 4, 2001
(86) PCT No.: PCT/FR01/01015

(30) Foreign Application Priority Data
Apr. 6, 2000 (FR) 00/04421

(51) Int. Cl. A61F 2/32

(52) U.S. Cl. 623/22.17; 623/22.19

(57) ABSTRACT

Acetabular insert comprising a spherical head arranged at one end of a medullary stem and mounted with articulation inside a core, which is itself mounted with articulation inside an acetabular component. The spherical head is made of zirconia and the core comprises an inner cup consisting of an alumina cup on which is immobilized a polyethylene cup overmolded on the alumina cup. In addition, the spherical head is configured so as to be able to be engaged without stress in the cup and be retained directly and exclusively by the latter.

### BACKGROUND OF THE INVENTION

The present invention relates to an acetabular insert for a total hip prosthesis.

Deterioration of the acetabular cavity frequently entails implantation of a total hip prosthesis. Such a prosthesis comprises, on the one hand, a femoral element with a medullary stem which is intended to be fixed to the bone inside the femur and whose free end is provided with a spherical head, and, on the other hand, an acetabular element intended to be engaged and fixed in the acetabular cavity. The acetabular element delimits a spherical cavity which receives, with the possibility of pivoting, the spherical head of the femoral element.

### DESCRIPTION OF THE PRIOR ART

An acetabular element known in particular from document FR 2 710 836 consists of a core made of a material which promotes sliding, in most cases of high-density polyethylene, which core delimits the cavity in which the spherical head is mounted with pivoting. This polyethylene core is inserted into a metal cup fixed in the acetabular cavity. Most of the movements are obtained from pivoting of the spherical head inside the core. However, in some movements of great amplitude, the polyethylene cup can also pivot inside the metal cup. It is thus possible to obtain dual mobility of the spherical head with respect to the acetabular cavity.

Patent application FR 99 08 194 describes a total hip prosthesis in which the contact surfaces of the head and of the core are made of hard-friction materials.

### SUMMARY OF THE INVENTION

The object of the invention is to make available a total hip prosthesis with which it is possible to obtain dual mobility, on the one hand of the spherical head with respect to the core, and on the other hand of the core with respect to the acetabular cavity, and which limits the risks of dislocation and has a long useful life.

To this end, in the prosthesis to which it relates, of the type comprising a spherical head arranged at one end of a medullary stem, which is intended to be fixed to the bone inside the femur, and mounted with articulation inside a core, which is itself mounted with articulation inside an acetabular component consisting, for example, of a metal cup fixed in the acetabular cavity, the spherical head is made of zirconia and the core comprises an inner cup which, intended for the fitting of the spherical head, consists of an alumina cup on which is immobilized a polyethylene cup overmolded on the alumina cup, and the spherical head is configured so as to be able to be engaged without stress in the inner cup and to be able to be retained directly in this cup without action of the outer polyethylene cup.

The retention of the head in the cup is ensured by the inner cup, without the use of any elastic retention by the outer polyethylene cup.

In order to ensure a maximum bearing surface of the spherical head and to ensure the retention of the latter, the alumina cup comprises, starting from its bottom and in the direction of its opening, a spherical surface portion with the same radius as that of the head of the femoral element, extending over more than a hemisphere, and continued below its equatorial plane by a cylindrical portion with a diameter less than that of the head of the femoral element.

In order to permit the introduction of the head inside the alumina cup, the head comprises, on part of its surface, at least one zone of material recessed relative to the spherical surface, corresponding to a reduction in diameter to a value less than the diameter of the entrance of the inner cup, in order to allow the head to pass without force into the entrance thereof, in a position in which the axis of fitting of the head on the neck of a femoral stem is inclined with respect to the axis of the entrance.

According to another characteristic of the invention, the polyethylene cup comprises a return piece at least partially covering the edge of the alumina cup delimiting the opening thereof. This return piece of the polyethylene cup serves as an abutment for the neck of the femoral stem in the event of substantial inclinations thereof.

So as not to limit excessively the movements of the neck of the femoral stem, the return piece of the polyethylene cup comprises an entrance of general conical shape widening toward the outside.

In order to ensure excellent holding of the polyethylene cup on the alumina cup, the latter comprises a certain number of flat parts formed on its periphery and comprises at least one annular and peripheral groove perpendicular to its axis.

According to another characteristic of the invention, the head made of zirconia comprises a frustoconical recess with a conicity of the order of 4°, allowing it to be fitted on a neck of corresponding conicity belonging to a femoral stem. This low conicity makes it possible to generate substantial frictional forces opposing the expulsion of the head of zirconia from the support of the femoral neck, without for this purpose requiring a substantial axial force of impaction, which would be prejudicial to the good mechanical stability of the head of zirconia.

This insert can be mounted in a metal cup fixed in the acetabular cavity by brackets or studs or engaged with force in this cavity. Such a prosthesis has the advantages of dual mobility and also the advantages of hard friction between zirconia and alumina, presenting a low degree of wear and preventing the release of polyethylene which happens when the head of the prosthesis is engaged in a cup made of polyethylene. The result of this is that the prosthesis has a useful life which is increased considerably compared to a prosthesis comprising metal-polyethylene friction or alumina-polyethylene friction, while at the same time avoiding the drawbacks resulting from the release of the polyethylene particles inside the body.

### BRIEF DESCRIPTION OF THE DRAWING

In any case, the invention will be fully understood with the aid of the following description in which reference is made to the attached diagrammatic drawing which shows, by way of nonlimiting example, an embodiment of this insert:

FIG. 1 is an exploded perspective view of the various constituent parts of this insert;

FIG. 2 is a sectional view of the core along the line II-II in FIG. 1;

FIG. 3 is a sectional view during introduction of the head of the prosthesis inside the core;

FIG. 4 is a sectional view of the prosthesis in the implanted position.

### DESCRIPTION OF A PREFERRED EMBODIMENT

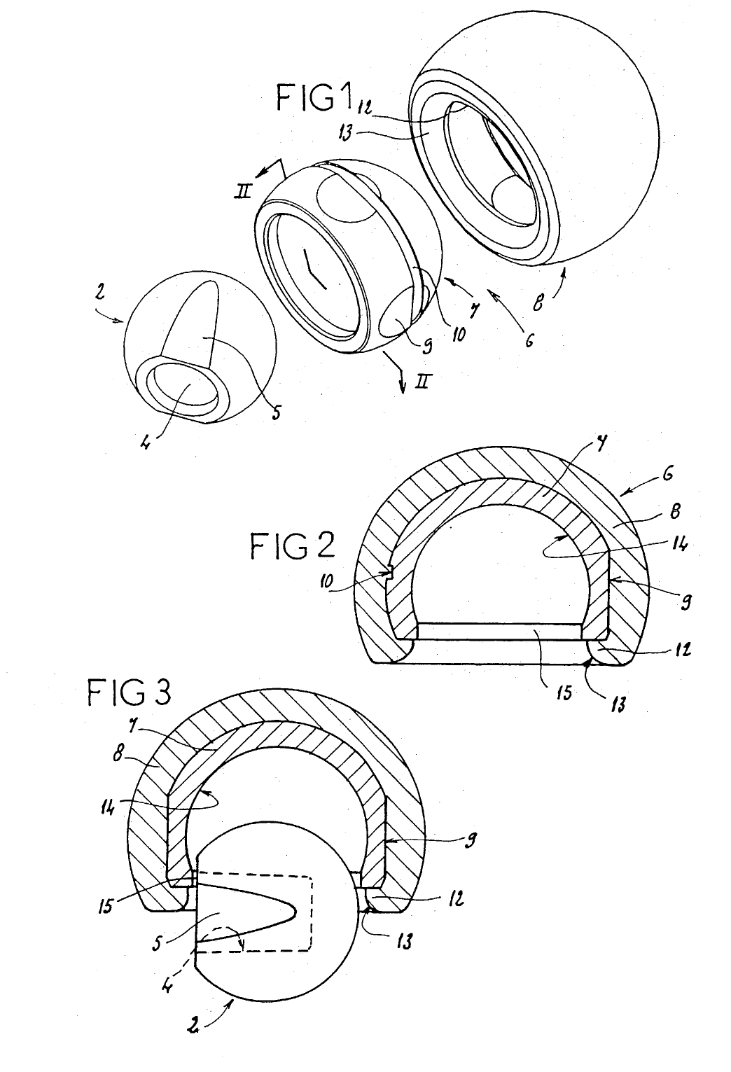

The prosthesis shown in the drawing comprises a spherical head 2 mounted at one end of a medullary stem (not shown in the drawing) intended to be fixed to the bone inside the femur. The head 2 is mounted on the medullary stem by way of a conical neck 3 integral with the stem, with a cone of conicity of the order of 4°, engaged in a recess 4 in the head 2. On its outer face, the head 2 has two zones 5 of recessed material arranged symmetrically and centered on a plane perpendicular to the entrance of the recess 4. The head 2 is intended to be mounted with articulation inside a core 6 comprising an alumina cup 7 on which a cup 8 made of high-density polyethylene is overmolded. FIG. 1 is a highly diagrammatic and theoretical view insofar as the cup 8 does not exist as such and independently, since it is overmolded on the cup 7. The cup 7, which is made of alumina, comprises, on its outer face, several flat parts 9 and a peripheral annular groove 10. The flat parts 9 and the groove 10 aim to ensure excellent blocking of the polyethylene cup 8 on the alumina cup 7. The polyethylene cup has a return piece 12 partially covering the edge of the cup 7 delimiting the opening thereof. The return piece 12 comprises a conical entrance 13 widened toward the outside.

The alumina cup comprises, starting from its bottom and in the direction of its entrance or opening, a spherical surface portion 14 with the same radius as that of the head 2 of the femoral element, extending over more than a hemisphere, and continued by a cylindrical portion 15 with a diameter less than that of the head of the femoral element.

It is however possible to introduce the head 2 of zirconia inside the alumina cup 7 by virtue of the zones of recessed material which the head comprises. This is because these zones of recessed material afford a cross section which is less than the cross section of the entrance of the cup 7 in its, cylindrical portion 15. After engagement in a position in which the opening of the frustoconical recess 4 is substantially perpendicular to the entrance of the cup 7, the head can be swung round before fitting the conical neck 3. Once this fitting has been done, the head of the prosthesis can no longer be removed from the alumina cup 7.

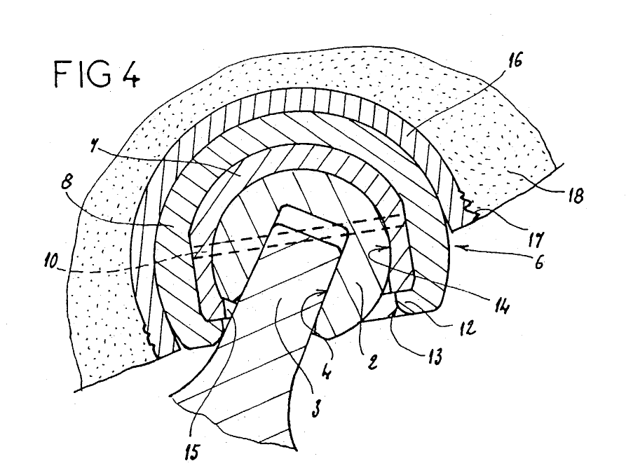

This insert can be mounted in a metal cup 16, for example a cup engaged with force and maintained by virtue of notches 17 in the bone 18 in which the acetabular cavity is formed. It will be noted that, as is shown in FIG. 4, the return piece 12 of the polyethylene cup 8 forms an abutment for the neck 3 of the medullary stem when movements of great amplitude are executed.

As will be clear from the foregoing, the invention affords a great improvement to the existing technique by making available an acetabular insert for a total hip prosthesis whose useful life is very long because the friction takes place between the zirconia of the spherical head and the alumina of a cup, the mobility being increased by virtue of the possibility of pivoting of the polyethylene cup inside the metal cup 16 fixed in the pelvis.

It goes without saying that the invention is not limited to the single embodiment of this acetabular insert which has been described above by way of example, and instead it encompasses all variants thereof. Thus, in particular, the means for blocking the polyethylene cup on the alumina cup could be different and formed not by flat parts and by a groove, but instead by hollowed cavities, the return piece of the polyethylene cup could have a different shape, the alumina cup could have a bevel at the level of its entrance, or the polyethylene cup could be mounted in a metal cup fixed otherwise than by force in the acetabular cavity, without thereby departing from the scope of the invention.

1. An acetabular insert for a total hip prosthesis of the type comprising a spherical head arranged at one end of a medullary stem, which is intended to be fixed to the bone inside the femur, and mounted with articulation inside a core, which is itself mounted with articulation inside an acetabular component consisting, for example, of a metal cup fixed in the acetabular cavity, wherein the spherical head is made of zirconia and the core comprises an inner cup which, intended for the fitting of the spherical head, consists of an alumina cup on which is immobilized a polyethylene cup overmolded on the alumina cup, and wherein the spherical head is configured so as to be able to be engaged without stress in the inner cup and to be able to be retained directly in this cup.

2. The acetabular insert as claimed in claim 1, wherein the alumina cup comprises, starting from its bottom and in the direction of its opening, a spherical surface portion with the same radius as that of the head of the femoral element, extending over more than a hemisphere, and continued below its equatorial plane by a cylindrical portion with a diameter less than that of the head of the femoral element.

3. The acetabular insert as claimed in either of claims 1 and 2, wherein the head comprises, on part of its surface, at least one zone of material recessed relative to the spherical surface, corresponding to a reduction in diameter to a value less than the diameter of the entrance of the inner cup, in order to allow the head to pass without force into the entrance thereof, in a position in which the axis of fitting of the head on the neck of a femoral stem is inclined with respect to the axis of the entrance.

4. The acetabular insert as claimed in one of claims 1 through 3, wherein the polyethylene cup comprises a return piece at least partially covering the edge of the alumina cup delimiting the opening thereof.

5. The acetabular insert as claimed in claim 4, wherein the return piece of the polyethylene cup comprises an entrance of general conical shape widening toward the outside.

6. The acetabular insert as claimed in one of claims 1 through 5, wherein the alumina cup comprises a certain number of flat parts formed on its periphery.

7. The acetabular insert as claimed in one of claims 1 through 6, wherein the alumina cup comprises at least one annular and peripheral groove perpendicular to its axis.

8. The acetabular insert as claimed in one of claims 1 through 7, wherein the head made of zirconia comprises a frustoconical recess with a conicity of the order of 4°, allowing it to be fitted on a neck of corresponding conicity belonging to a femoral stem.
